(19) United States
(12) Patent Application Publication
(10) Pub. No.: US 2021/0137992 A1
(43) Pub. Date: May 13, 2021

(54) HYPOXIA-INDUCIBLE FACTOR-2A AS A TARGET IN PREVENTION/TREATMENT OF PARKINSON'S DISEASE

(71) Applicant: QIngdao University, Shandong (CN)

(72) Inventors: Junxia Xie, Shandong (CN); Jun Wang, Shandong (CN); Manman Xu, Shandong (CN)

(21) Appl. No.: 17/091,823

(22) Filed: Nov. 6, 2020

(30) Foreign Application Priority Data
Nov. 8, 2019 (CN) 201911087899.4

(51) Int. Cl. A61K 35/30; C12N 5/079

(52) U.S. Cl. CPC A61K 35/30; C12N 2509/00; C12N 2502/081; C12N 5/0622

(57) ABSTRACT

The present invention provides hypoxia-inducible factor-2α (HIF-2α) as a target in the prevention/treatment of Parkinson's disease (PD). It is the first time to report the target that illustrated mechanisms of iron metabolism in astrocytes, accordingly to confirm the similarities and differences of iron traffic between astrocytes and neurons, leading to indicated the iron source of iron deposition in dopaminergic (DA) neurons and make sure the cause of iron is unevenly distributed in different brain regions, as well as the effects of glias on the role of iron traffic in neurons. It is HIF-2α, regulates the iron traffic in astrocytes. Therefore, not only a new action target HIF-2α is provided for preventing/treating PD iron deposition, but also a brand-new research thought and experimental evidence are provided for the iron transport mechanism of the astrocytes as the high iron source of nerve cells.

### TECHNICAL FIELD

The present invention belongs to the field of nerve cells and, in particular, relates to use of confirm hypoxia-inducible factor-2a as a target in the prevention/treatment of Parkinson's disease.

### BACKGROUND

Parkinson's disease (PD) is a neurodegenerative disorder characterized in its late phase by the sustained loss of dopaminergic neurons in the substantia nigra pars compacta (SNpc). Clinically PD is characterized by bradykinesia, rigidity, rest tremor and postural instability. Although multiple factors such as genetic mutation, environmental factors, and aging might be involved in PD pathogenesis, its underlying mechanisms have not been revealed yet. In both animal PD models and PD patients, selective deposition of iron in the substantia nigra (SN) has been observed, while no iron content increases have been observed in other brain regions. Most current studies using PD models focused on iron deposition in dopaminergic neurons. However, a variety of gliocytes also plays important roles in cellular iron. In particular, astrocytes play an essential role in regulating brain iron homeostasis and preventing damage from iron-mediated oxidative stress.

Hypoxia-inducible factors (HIFs) are involved in regulating iron homeostasis and are heterodimers consisting of two helix-loop-helix-containing subunits: an oxygen-responsive regulatory subunit and a constitutively expressed HIF-1β, also known as the aryl-hydrocarbon receptor nuclear translocator (ARNT). Three regulatory HIF subunits have been characterized: HIF-1α, HIF-2α and HIF-3α. HIF-1α is expressed ubiquitously, whereas HIF-2α expression appears to be restricted to certain tissues. The expression patterns and functional properties of HIF-3α remain to be elucidated. Binding of HIF-2α to the promoter region of the HREs of DMT1 and FPN1 increases their expression Previous studies on HIF-1α and HIF-2α primarily focused on their regulation of iron metabolism in the small intestine, but less is known about their functions in the central nervous system. In the brain, the study of astrocytes and HIF mainly focuses on stroke, and HIF regulates VEGF expressed by the astrocytes, which is the main component of the blood-brain barrier that regulate the permeability of the blood-brain barrier through HIF, and there is no related report about the regulation of iron metabolism in the astrocytes by HIF.

Most current studies using PD models focused on neuroprotective effects on astrocytes cross-talk with dopaminergic neurons, such as astrocytes release BDNF and GDNF to protect dopaminergic neurons, the anti-oxidative stress effects of SOD, etc. Therefore it is unclear whether HIF-1α or HIF-2α plays a more critical role in astrocytic iron traffic, as well as the mechanism of iron metabolism in astrocytes. There is no studies to indicate. It is unclear whether astrocytes contribute to iron metabolism, and whether the iron which accumulates in SN neurons in PD comes from astrocytes. Those questions urgently need to be studied.

Therefore, it is curtail to search for a target to illustrate mechanisms of iron metabolism in astrocytes, accordingly to confirm the similarities and differences of iron traffic between astrocytes and neurons, leading to indicated the iron source of iron deposition in DA neurons and make sure the cause of iron is unevenly distributed in different brain regions, as well as the effects of glias on the role of iron traffic in neurons, in order to prevent/treat with PD.

### SUMMARY

The present invention provides use of HIF-2α as an action target in prevention/treatment of PD. In this solution, the action target HIF-2α of iron metabolism in astrocyte is found by research, and the action target for prevention/treatment of PD is found at the cellular level, which provides new research thought and experimental evidences for scientific problems that need to be solved urgently, such as the source of mesencephalon neuron iron in PD iron deposition and the iron transport action of the astrocytes.

In order to achieve the aforementioned objective, the present invention provides HIF-2α as an action target in prevention/treatment of the PD.

Preferably, it includes the following step:

respectively treating primary-cultured mesencephalon astrocytes and primary-cultured mesencephalon ventral neurons with 6-OHDA, respectively detecting the protein expressions of HIF-1α and HIF-2α in the extracted total proteins, and respectively comparing with the protein expressions of HIF-1α and HIF-2α in a group not treated with 6-OHDA, wherein HIF-1α and HIF-2α are detected in the astrocytes activated with 6-OHDA, and are not detected in the non-activated mesencephalon ventral neurons, so that it is confirmed that in a PD cell model prepared with 6-OHDA, 6-OHDA activates HIF-1α and HIF-2α in the primary-cultured mesencephalon astrocytes;

using Bay87-2243 as HIF-1α specific inhibitor to act on the primary-cultured mesencephalon astrocytes, then using 6-OHDA for co-action, and observing the change in the protein expression of HIF-1α in the astrocytes and the change in the protein expression of DMT1 (the iron transport-in protein Divalent Metal Transporter 1) and FPN1 (iron transport-out protein) in the astrocytes after HIF-1α activation is inhibited;

Using HIF-2α translation inhibitor as HIF-2α specific inhibitor to act on the primary-cultured mesencephalon astrocytes, then using 6-OHDA for co-action, and observing the change in the protein expression of HIF-2α in the astrocytes and the change in the protein expressions of DMT1 and FPN1 in the astrocytes after HIF-2α activation is inhibited; Screening based on the aforementioned changes in the protein expression in the primary-cultured mesencephalon astrocytes and primary-cultured mesencephalon ventral neurons, to obtain the hypoxia-inducible factor 2a as an action target for prevention/treatment of PD.

Preferably, the activation of HIF-2α in the primary-cultured astrocytes can cause up-regulation of the protein expressions of DMT1 and FPN1, and the inhibition of HIF-2α in the astrocytes can also inhibit the up-regulation of the protein expression of DMT1 and FPN1.

Preferably, the HIF-1α inhibitor inhibits the protein expression of HIF-1α in the primary-cultured astrocytes, but does not inhibit the up-regulation of the protein expression of DMT1 and FPN1 in the primary-cultured astrocytes as induced by 6-OHDA.

Preferably, the primary-cultured astrocytes and the primary-cultured mesencephalon ventral neurons are respectively treated with 10 μM of 6-OHDA and then inoculated in a six-well plate pre-coated with polylysine at a density of 1.5×10⁵/ml, and then 24 h later the total protein is extracted respectively; and

pretreatment is conducted for 48 h or 24 h with 10 μM of the HIF-1α specific inhibitor Bay87-2243 or HIF-2α translation inhibitor, which is the HIF-2α specific inhibitor in connection with instructions of the inhibitors and a screen-time experiment, then added with 10 μM of 6-OHDA for co-action for 24 h, and then the total protein is extracted.

Preferably, the method for extracting and quantifying the total protein includes the following steps:

treating cells with 6-OHDA or the HIF-1α or HIF-2α specific inhibitor, blotting up the culture medium, washing with pre-chilled PBS, adding 100 μl of RIPA cell lysis buffer into each well, and lysing on ice for 0.5 h to obtain a lysate; and

collecting the lysate, then centrifuging at 4° C. and 12000 rpm for 20 min, and transferring the supernatant into a new EP tube; detecting the concentration of the extracted protein with a BCA protein quantitative kit, sub-packaging the protein according to 30 μg per serving, heating in a water bath at 95° C. for 5 min to denature the protein, and cryopreserving in a refrigerator at −80° C.

Preferably, the culture approach of the astrocytes includes: extracting and separating the primary mesencephalon astrocytes from a test animal, resuspending the precipitate, screening, then digesting with pancreatin, and terminating the digestion with a astrocyte complete medium, then adding the astrocyte complete medium for differential adhesion treatment, adjusting the cell concentration, then inoculating into a six-well plate pre-treated with polylysine, and stabilizing for 24-48 h for experiments;

the culture approach of the mesencephalon ventral neurons includes: extracting and separating the primary mesencephalon ventral neurons from a test animal, inoculating into a six-well plate pre-treated with polylysine, placing in a 5% CO₂ incubator at 37° C. for 5 days, and then using for experiments.

Preferably, the astrocyte complete medium consists of 200 ml of a DMEM/F-12(1:1) basic medium, 10% fetal bovine serum, 100 U/ml of penicillin and 0.1 mg/ml of streptomycin.

Preferably, a mesencephalon ventral inoculation medium consists of 200 ml of the DMEM/F-12(1:1) basic medium, 10% fetal bovine serum, 100 U/ml of penicillin and 0.1 mg/ml of streptomycin; and a mesencephalon ventral neuron complete medium consists of 200 ml of the DMEM/F-12(1:1) basic medium, 2% B27, 100 U/ml of penicillin and 0.1 mg/ml of streptomycin.

Preferably, the pathological feature of the Parkinson's disease is the loss of dopaminergic neurons in substantia nigra pars compacta, iron deposition of the Parkinson's disease is mainly concentrated in the dopaminergic neurons, and the iron deposition in the dopaminergic neurons leads to degeneration and death of the dopaminergic neurons, leading to the occurrence of the Parkinson's disease.

As compared with the prior art, the advantages and positive effects of the present invention are as follows.

1. In the present invention, PD cell model of the primary-cultured mesencephalon astrocytes and the primary-cultured VM neurons is utilized to clarify the difference of iron metabolism between astrocytes and neurons, wherein in the neurons, 6-OHDA treated with primary cultured VM neurons, and Iron regulatory protein 1 (IRP1) is activated, which up-regulates the expression of DMT1 and down-regulates the expression of FPN1, acceleration of iron transport-in and slowdown of iron transport-out, resulting in iron deposition in the neurons. That is, IRP1 plays a major role in regulating iron metabolism in the VM neurons. Moreover, 6-OHDA acts on the primary-cultured mesencephalon astrocytes, the protein expression of IRP1 does not change, and HIF-2α is activated, which enables up-regulation of DMT1 and FPN1 and acceleration of iron transport-in and transport-out, thereby accelerating the iron transport in the astrocytes. That is, HIF-2α plays a major role in regulating iron metabolism in the primary-cultured mesencephalon astrocytes.

2. In the present invention, a classical PD cell model is effectively used for the study of iron metabolism of PD, and especially of iron transport, which not only specifies the regulation mechanism of iron metabolism in the astrocytes, but also clarifies the role of the astrocytes as an “iron pump”, that is, iron is transported in by DMT1 and transported out by FPN1 in the astrocytes, and this process is regulated by HIF-2a. Also, the role of the astrocytes in iron deposition of PD is clarified, that is, 6-OHDA activates HIF-2α in the astrocytes, so that the protein expression of DMT1 and FPN1 is increased, the transport of iron into and out of cells is accelerated, and too much iron is transported into the neurons, which is the source of iron deposition in the neurons of PD.

3. The key point and emphasis of the present invention lies in that the present invention has found the source of high iron in the nerve cells for PD iron deposition, that is, the PD iron deposition is caused by the acceleration of iron transport in the astrocytes, and the core element for promoting the acceleration of iron transport in the astrocytes is HIF-2a. Therefore, by the research of this patent, not only a new action target HIF-2α is provided for preventing/treating PD iron deposition, but also a brand-new research thought and experimental evidences are provided for the iron transport mechanism of the astrocytes as the high iron source of the nerve cells. It has potential immeasurable social and economic values for research and development of drugs for preventing and treating PD.

4. In the present invention, during the study of transport of iron into the astrocytes, exogenous iron is given for 30 min, and real-time scanning is conducted to observe dynamic changes. It is found that iron will change the morphology and function of a cell after 30 min; during the study of transport of iron out from the astrocytes, exogenous iron should be given for 30 min first. If the time is too long, the real-time change of intracellular iron will change, which may be related to the own physiological repair mechanism of the cell; and then DFO is used for action for 30 min, real-time scanning is conducted to observe dynamic changes, and it is found that the fluorescence intensity is gradually increased.

### BRIEF DESCRIPTION OF THE DRAWINGS

FIG. 1 shows the changes of protein expressions of HIF-1α and HIF-2α in primary-cultured mesencephalon astrocytes that are treated with 10 μM of 6-OHDA for 24 h, as provided by embodiments of the present invention;

FIG. 2 shows the changes of protein expressions of HIF-1α and HIF-2α in primary-cultured VM neurons that are treated with 10 μM of 6-OHDA for 24 h, as provided by embodiments of the present invention;

FIG. 3 shows the changes of protein expression of HIF-1α in primary-cultured mesencephalon astrocytes that are pre-treated with 10 μM of Bay87-2243 for 48 h and then treated with 10 μM of 6-OHDA for 24 h, as provided by embodiments of the present invention;

FIG. 4 shows the changes of protein expression of DMT1 in primary-cultured mesencephalon astrocytes that are pre-treated with 10 μM of Bay87-2243 for 48 h and then treated with 10 μM of 6-OHDA for 24 h, as provided by embodiments of the present invention;

FIG. 5 shows the changes of protein expression of FPN1 in primary-cultured mesencephalon astrocytes that are pre-treated with 10 μM of Bay87-2243 for 48 h and then treated with 10 μM of 6-OHDA for 24 h, as provided by embodiments of the present invention;

FIG. 6, (A) shows the changes of protein expression of HIF-2α in primary-cultured mesencephalon astrocytes that are pre-treated with 10 μM of a HIF-2α inhibitor for 24 h and then treated with 10 μM of 6-OHDA for 24 h, as provided by embodiments of the present invention; (B) shows the changes of protein expression of DMT1 in primary-cultured mesencephalon astrocytes that are pre-treated with 10 μM of a HIF-2α inhibitor for 24 h and then treated with 10 μM of 6-OHDA for 24 h, as provided by embodiments of the present invention; and (C) shows the changes of protein expression of FPN1 in primary-cultured mesencephalon astrocytes that are pre-treated with 10 μM of a HIF-2α inhibitor for 24 h and then treated with 10 μM of 6-OHDA for 24 h, as provided by embodiments of the present invention;

FIG. 7 is a schematic diagram of transport of iron into (a) and out (b) of primary-cultured mesencephalon astrocytes, as provided by embodiments of the present invention.

### DESCRIPTION OF THE EMBODIMENTS

In order to introduce HIF-2α as an action target in the prevention/treatment of the PD more clearly and in detail, the technical solution in the embodiments of the present invention will be described clearly and completely below. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

### Example 1 Primary Culture of Astrocytes

1.1 Experimental Animals

Wistar suckling mice newly born in 24 h (provided by Animal Center of Qingdao Municipal Institute for Drug Control) were used as experimental animals for primary culture of astrocytes.

1.2 Experimental Reagents

DMEM/F-12 (1:1): a product available from Gibco;

Fetal bovine serum (FBS Gold): a product available from Gibco, of EU grade;

Polylysine, boric acid, sodium tetraborate: products available from Sigma;

Penicillin-streptomycin solution (100×) and Trypsin: products available from Beyotime Institute of Biotechnology, Haimen, Jiangsu.

1.3 Experimental Instruments

Clean bench: available from Suzhou Purification Equipment Co., Ltd.;

CO₂ incubator: a product available from Thermo Fisher Scientific, USA;

Ordinary centrifugal machine: available from ANHUI USTC Zonkia Scientific Instruments Co., LTD.;

Thermostat and oven: a product available from MEMMERF, German;

Refrigerators at −20° C. and 4° C.: a product available from Haier, Qingdao;

Ultra-low temperature refrigerator at −80° C.: a product available from Thermo Fisher Scientific, USA;

Autoclave: a product available from VARIOKLAV, Germany;

One-hundred-thousandth electronic balance: a product available from Shimadzu, Japan (AEL-40SM);

Dissecting microscope, inverted phase contrast microscope: a product available from Olympus, Japan;

Air bath constant-temperature shaker: available from Harbin Dongming Medical Instrument Factory.

1.4 Liquid Formulation

DMEM/F-12(1:1) Basic Medium:

1 package of DMEM/F12 powder was dissolved in 1000 ml of double distilled water, added with 2.438 g of NaHCO₃, adjusted until the pH was 7.2, sterilized by filtering through a 0.2 μm microporous membrane, sub-packaged, and stored at 4° C.

Astrocyte Complete Medium:

200 ml of a DMEM/F-12(1:1) basic medium, 20% fetal bovine serum, 100 U/ml of penicillin and 0.1 mg/ml of streptomycin were mixed evenly and stored at 4° C.

5× Borate Buffer:

0.254 g of boric acid and 0.476 g of sodium tetraborate were weighed, dissolved in double distilled water, then brought to a constant volume of 100 ml after complete dissolution, sterilized by filtering through a 0.22 μm microporous membrane, and stored at 4° C.

Polylysine:

polylysine was dissolved in a sterilized 5× borate buffer to obtain a stock solution with a concentration of 1 mg/ml, and then sub-packaged and stored at −20° C. When a non-glass product was coated, the polylysine solution was diluted to a working concentration (10 μg/ml) with a sterilized 1× borate buffer.

1.5 Culture and Screening of Astrocytes

The culture and isolation of the astrocytes were operated according to the literature, and the specific steps were as follows:

Treatment of Culture Plate and Culture Flask

Before cell inoculation, polylysine (10 μg/ml) was added into a 150 cm² cell culture flask (Corning) in such a manner that polylysine completely covered the bottom of the flask, and placed in a 5% CO₂ incubator at 37° C. overnight. The remaining polylysine solution was blotted up. The flask was washed with autoclaved double distilled water for three times, and irradiated with ultraviolet rays on the clean bench for 0.5 h for later use.

Acquisition, passage, and inoculation culture of primary astrocytes:

Wistar suckling mice born within 24 h were soaked in 75% alcohol for disinfection. The mouse was killed by cervical dislocation, and the head of the mouse was taken. The head was cut open with Ophthalmic scissors on both sides of the head of the suckling mouse along the upper margin of ears to minimize the damage to the brain tissue. The whole brain was stripped with ophthalmic forceps, and placed in a plate of DMED/F12 basic medium pre-chilled at 4° C. Thereafter, all operations were completed on ice to reduce cell death. The cerebellum and olfactory bulb sites were removed with corneal forceps under a dissecting microscope. Meninges and blood vessels were carefully peeled off, put in a new plate of sterile DMED/F12 basic medium. The brain tissue was chopped with the ophthalmologic scissors, and pipetted up and down gently with a sterilized Pasteur dropper, a pipette tip with an aperture diameter of 1 mm, and a pipette tip with an aperture diameter of 0.5 mm until the tissue mass was completely dissipated. The unbroken tissue mass was removed by filtering through a 200-mesh cell sieve. The filtrate was transferred into a centrifuge tube, and centrifuged at 1000 rpm for 5 min. The supernatant was discarded, and the precipitate was resuspended in the astrocyte complete medium containing 20% fetal bovine serum and double antibodies. According to a density of about 5 brains/flask (20 ml), the cell suspension was inoculated into a 150 cm² cell culture flask treated with polylysine. The cell culture flask was placed in a 5% CO₂ incubator at 37° C. First, the culture flask was turned upside down, subjected to differential attachment for 1 hour, and then turned upright. Half of the medium was replaced once 24 h later, and then medium replacement was conducted every three days.

After 7-14 days of cell culture, it was observed that the cells at the bottom layer were grown to confluence, grown into dense monolayer cells, which can be used for screening the astrocytes. The cell culture flask was placed in the air bath constant-temperature shaker, and shaken vigorously at 37° C. and 240 rpm for 16-18 h. The supernatant was discarded. The cells at the bottom layer were rinsed once with the serum-free DMEM/F12 basic medium, and digested with the added 0.25% pancreatin at 37° C. for 1-5 min, and then the digestion was terminated with the astrocyte complete medium. The cell suspension was centrifuged at 1000 rpm for 5 min, and the supernatant containing enzymes was discarded. The cells were washed once again, added with the astrocyte complete medium and subjected to differential attachment for 30 min. The cells were then resuspended, and the cell density was adjusted. The cells were subcultured at about 5×10⁴/cm². After 2 generations, the cells were inoculated into a 6-well plate treated with polylysine, and stabilized for 24-48 h before being used for experiments.

### Example 2 Primary Culture of Ventral Mesencephalon (VM) Neurons

2.1 Experimental Animals

Wistar mice (provided by Animal Center of Qingdao Municipal Institute for Drug Control), which were pregnant for 14 days, were selected as the experimental animals, which lived at room temperature of 20° C. under an illumination condition of day-night cycle of 12:12 hours and had free access to feedstuff and drinking water.

2.2 Experimental Reagents

DMEM/F-12 (1:1): a product available from Gibco;

B27: a product available from Gibco;

Fetal bovine serum (FBS Gold): a product available from Gibco, of EU grade;

Polylysine, boric acid, sodium tetraborate: products available from Sigma;

Penicillin-streptomycin solution (100×) and Trypsin: products available from Beyotime Institute of Biotechnology, Haimen, Jiangsu.

2.3 Experimental Instruments

Clean bench: available from Suzhou Purification Equipment Co., Ltd.;

CO₂ incubator: a product available from Thermo Fisher Scientific, USA;

Ordinary centrifugal machine: available from ANHUI USTC Zonkia Scientific Instruments Co., LTD.;

Thermostat and oven: a product available from MEMMERF, German;

Refrigerators at −20° C. and 4° C.: a product available from Haier, Qingdao;

Ultra-low temperature refrigerator at −80° C.: a product available from Thermo Fisher Scientific, USA;

Autoclave: a product available from VARIOKLAV, Germany;

One-hundred-thousandth electronic balance: a product available from Shimadzu, Japan (AEL-40SM);

Dissecting microscope, inverted phase contrast microscope: a product available from Olympus, Japan;

2.4 Liquid Formulation

DMEM/F-12(1:1) Basic Medium:

1 package of DMEM/F12 powder was dissolved in 1000 ml of double distilled water, added with 2.438 g of NaHCO₃, adjusted until the pH was 7.2, sterilized by filtering through a 0.2 μM microporous membrane, sub-packaged, and stored at 4° C.

Inoculation Medium of VM Neurons:

200 ml of a DMEM/F-12(1:1) basic medium, 10% fetal bovine serum, 100 U/ml of penicillin and 0.1 mg/ml of streptomycin were mixed evenly and stored at 4° C.

Complete Medium of VM Neurons:

200 ml of a DMEM/F-12(1:1) basic medium, 2% B27, 100 U/ml of penicillin and 0.1 mg/ml of streptomycin were mixed evenly and stored at 4° C.

5× Borate Buffer:

0.254 g of boric acid and 0.476 g of sodium tetraborate were weighed, dissolved in double distilled water, then brought to a constant volume of 100 ml after complete dissolution, sterilized by filtering through a 0.22 μm microporous membrane, and stored at 4° C.

Polylysine:

polylysine was dissolved in a sterilized 5× borate buffer to obtain a stock solution with a concentration of 1 mg/ml, and then sub-packaged and stored at −20° C. When a non-glass product was coated, the polylysine solution was diluted to a working concentration (10 μg/ml) with a sterilized 1× borate buffer.

2.5 Primary Culture of VM Neurons

1 day before inoculation, 6 well plats were treated with 10 μg/ml or 20 μg/ml of polylysine, and was placed in an incubator at 37° C. overnight. The liquid was blotted up, the lower chamber was washed with sterile triple distilled water for three times, and air dried in the clean bench. The pregnant Wistar Rat was anesthetized with chloral hydrate (400 mg/kg, 8%, about 1 ml/200 mg) on the 14th day of pregnancy, then the abdominal skin was sterilized with 75% alcohol, and cut open along an abdominal midline to fully expose the abdominal cavity. The uterus (embryo sacs connected in series) was completely taken out, and then placed in a glass dish accommodating a pre-chilled D-hank'S solution. The envelope was cut open to get a fetal mouse out into a new D-hank'S solution. The embryo was separated, and the telencephalon was removed with fibrous curved ophthalmologic forceps under the dissecting microscope. The mesencephalon portion was taken out and put into a new D-hanks solution. The remaining brain tissue mass was cropped to remove the meninges and leave the butterfly-shaped ventral mesencephalon, which was transferred into a new D-hanks solution. A large brain tissue mass was broken with a sucker, and then gently pipetted up and down with a pipette tip until the tissue mass was completely separated and form a suspension of individual cells. The cell suspension was transferred into a large centrifuge tube, placed into a centrifugal machine, and centrifuged at 1000 rpm/5 min for 5 min. The supernatant was discarded carefully. The precipitate was resuspended by adding a proper amount of an inoculation fluid, and pipetted up and down repeatedly until the tissue mass was completely dissipated. 10 μl of the cell suspension was taken and diluted by 10 times, and then stained with trypan blue. The cells were counted under an inverted microscope. The stained cells were dead cells. The cell concentration was adjusted to 1×10⁶/ml by adding an appropriate amount of the inoculation fluid. Then the cells were inoculated into a 6-well plate pre-treated with polylysine. After the cells were incubated in a 5% CO₂ incubator at 37° C. for 18 h, the medium was replaced with a medium containing 2% B27. Then medium replacement was conducted every three days, and the cells could be used for experiment after 5 days.

### Example 3 Activation of HIF-1α and HIF-2α and Protein Expression of DMT1 and FPN1 as Detected by Western Blot

3.1 Experimental Reagents

6-hydroxydopamine (6-OHDA) and ascorbic acid: products available from Sigma.

HIF-2α translation inhibitor: a product available from Millipore

Bay87-2243: a product available from Selleck, used as a HIF-1α specific inhibitor

Acrylamide, benzyl sulfonamide (PMSF): products available from Genview.

Ammonium persulfate (APS), Tween-20, sodium dodecyl sulfate (SDS, of electrophoresis grade), NonidetP40, pepstatin, aprotinin, leupeptin, sodium deoxycholate, rabbit anti-β-actin antibody, and HRP-tagged goatanti-rabbit IgG: products available from Sigma.

N′,N′-methylenebisacrylamide: a product available from Bebco.

Aminoacetic acid (Glycine, of electrophoresis grade), trihydroxy aminomethane (Tris), Tris-Base, Tris-HCl and ethylenediamine tetra acetic acid (EDTA): products available from Solarbio.

PDVF membrane, and ECL chemiluminescence kit: products available from Millipore.

RIPA lysis buffer (medium), BCA protein quantitative kit, color-pre-stained protein molecular weight standard, N,N,N′,N′-tetramethylethylenediamine (TEMED): products available from Beyotime Institute of Biotechnology, Haimen, Jiangsu.

The primary antibody: DMT1 and FPN1 antibodies were products available from ADI, the HIF-1α antibody was a product available from Sigma, the HIF-2α antibody was a product available from Novus Biologicals, and a β-actin antibody was purchased from Bioss, Beijing.

The secondary antibody: the HRP-tagged goat anti-rabbit IgG, which was purchased from Santacruz.

3.2 Commonly Used Instruments

Electrophoresis chamber Mini-VE, electroporation instrument (wet portion) Trans-Blot, electrophoresis instrument Power-Pac200: available from BIO-RAD, USA.

Ultra-pure water machine: available from Millipore, USA.

Spectrophotometer, Desktop Low Temperature High Speed Centrifuge (5417R): available from Eppendorf, Germany.

The UVP gel imaging system was available from USA.

3.3 Formulation of Drugs as Used

Ascorbic Acid Solution:

ascorbic acid was dissolved in 0.9% normal saline to obtain a stock solution with a concentration of 200 μg/ml.

6-OHDA:

0.2056 mg of 6-OHDA was taken and dissolved in a ascorbic acid solution to obtain a 1 mM stock solution, and the stock solution was diluted with a serum-free medium to the concentration of a working solution when in use.

HIF-2α Translation Inhibitor (CAS 882268-69-1):

0.5746 mg of the inhibitor was taken and dissolved in 2 ml of DMSO to obtain a 1 mM stock solution, and the stock solution was diluted with the serum-free medium to the concentration of a working solution when in use. The HIF-2α translation inhibitor was a HIF-2α specific inhibitor.

Bay87-2243:

1 mg of Bay87-2243 was taken, added with 1.9028 ml of ethanol, and dissolved in a water bath at 37° C. to obtain a 1 mM stock solution, and the stock solution was diluted with the serum-free medium to the concentration of a working solution when in use. Bay87-2243 was a HIF-1α specific inhibitor.

3.4 Antibodies as Used

The primary antibody: the titer of rabbitanti-DMT1 and rabbitanti-FPN1 was 1:800, and the titer of rabbitanti-HIF-1α and rabbitanti-HIF-2α was 1:1000.

The titer of the β-actin antibody was 1:10000.

The secondary antibody: HRP-tagged goat anti-rabbit IgG with a titer of 1:10000.

3.5 Formulation of Commonly-Used Liquid

PMSF:

1.74 mg of PMSF was dissolved in 1 ml of isopropanol to obtain a stock solution with a concentration of 100 mmol/L, and the stock solution was stored at −20° C.

Cell Lysis Buffer:

50 mmol/L of Tris pH 7.5, 1% Nonidet40, 150 mmol/L of NaCl, 0.5% sodium deoxycholate (with protection from light), 1 mmol/L of EDTA, 10 mmol/L of PMSF, pH 7.5, were filtered and stored at 4° C. with protection from light Immediately before use, 987 μl of them was taken, and added with 10 μl of 100 mM PMSF, 1 μl of pepstatin (1 μg/ml), 1 μl of aprotinin (1 μg/ml), and 1 μl of leupeptin (1 μg/ml).

Mixed solution of 30% acrylamide (100 ml):

29 g of acrylamide and 1 g of N,N′-methylenebisacrylamide were dissolved in ultrapure water with a total volume of 60 ml, heated to 37° C. for dissolution, supplemented with water to a final volume of 100 ml, pH<7.0, filtered and stored at 4° C. with protection from light.

10% APS:

0.1 g of APS was added with 1 ml of ultrapure water and stored at 4° C., and it was prepared immediately before use.

10% SDS:

10 g SDS was dissolved in 80 ml ultrapure water, heated and dissolved, then brought to a constant volume of 100 ml, and stored at room temperature.

1.5M Tris-Cl (pH 8.8):

18.171 g of Tris was weighed and dissolved in 90 ml of ultrapure water, adjusted with concentrated HCl until the pH value was 8.8, and brought to a constant volume of 100 ml.

0.5M Tris-Cl (pH 6.8):

6.057 g of Tris-Cl was weighed and dissolved in 90 ml of ultrapure water, adjusted with concentrated HCl until the pH value was 6.8, and brought to a constant volume of 100 ml.

10× Electrophoresis Buffer:

30 g of Tris, 144 g of glycine, and 10 g of SDS were respectively weighed, and brought to a constant volume of 1000 ml with ultrapure water.

10× Membrane Transfer Buffer:

30 g of Tris and 144 g of glycine were respectively weighed, and brought to a constant volume of 1000 ml with ultrapure water.

1× Membrane Transfer Buffer:

100 ml of the 10× membrane transfer buffer, 200 ml of methanol, and 700 ml of ultrapure water were respectively weighed, and the pH value was adjusted to 8.3.

10×TBST Solution:

24.2 g of Tris, 80 g of NaCl, 0.375 g of SDS, 138 ml of 1.0M HC, and 10 ml of Tween-20 were respectively weighed, and brought to a constant volume of 1000 ml with ultrapure water.

Blocking Buffer:

10% skimmed milk powder was formulated with 1×TBST.

Bay87-2243(8 mg/ml):

immediately before use, it is diluted to a working solution concentration of 10 μM with a serum-free DMEM/F12 basic medium not containing the double antibodies.

6-OHDA:

immediately before use, it is diluted to a working concentration of 10 μM with the serum-free DMEM/F12 not containing the double antibodies or a DMEM high-sugar basic medium.

HIF-2α Translation Inhibitor:

immediately before use, it is diluted to a working concentration of 10 μM with a serum-free DMEM/F12 basic medium not containing the double antibodies.

3.6 Cell Inoculation and Drug Treatment

Inoculation and Drug Treatment of Astrocytes

The astrocytes were inoculated into a six-well plate pre-coated with polylysine at a density of 1.5×10⁵/ml. In order to detect the activation of HIF-1α and HIF-2α by 6-OHDA and the corresponding protein expression of DMT1 and FPN1, the cells were divided into groups and treated with drugs. After being treated with 10 μM of 6-OHDA for 24 h, the cells were collected to extract the total protein.

In order to test the influence of inhibiting the activation of HIF-2α on the protein expression of DMT1 and FPN1 in the astrocytes, the cells were pre-treated with 10 μM of the HIF-2α translation inhibitor for 24 h, and then added with 10 μM of 6-OHDA for co-action for 24 h. Then the total protein was extracted.

In order to test the influence of inhibiting the activation of HIF-1α on the protein expression of DMT1 and FPN1 in the astrocytes, the cells were pre-treated with 10 μM of Bay87-2243 for 48 h, and then added with 10 μM of 6-OHDA for co-action for 24 h. Then the total protein was extracted.

The activation and inhibition of HIF-1α and HIF-2α by 6-OHDA and the corresponding protein expression of DMT1 and FPN1 were detected by Western blots.

Inoculation and Drug Treatment of VM Neurons

In order to detect the activation of HIF-1α and HIF-2α in the VM neurons by 6-OHDA, the cells were divided into groups and treated with drugs. After being treated with 10 μM of 6-OHDA for 24 h, the cells were collected to extract the total protein.

3.7 Extraction and Quantification of Total Protein

After drug treatment of the cells, the medium was completely blotted up, the cells were washed with pre-chilled PBS for three times, and 100 μl of the cell lysis buffer: RIPA lysis buffer (strong) was added to each well, and the cells were lysed on ice for 0.5 h.

The lysate was collected and centrifuged at 12000 rpm and 4° C. for 20 min, and the supernatant was transferred into a new Eppendorf tube.

The concentration of the extracted protein was detected by the BCA protein quantitative kit. The protein was sub-packaged according to 30 μg per serving, brought to the same volume with 0.01M PBS, then added with a 4× loading buffer and mixed well, subjected to a water batch above 95° C. for 5 min to denature the protein, and cryopreserved in the refrigerator at −80° C.

3.8 Sodium Dodecyl Sulfate-Polyacrylamide (SDS-PAGE) Gel Electrophoresis and Membrane Transfer

8% separating gel (10 ml):

2.16 ml of the mixed solution of 30% acrylamide, 2 ml of 3 mol/Tris-HCl (pH 8.8), 80 μl of 10% SDS, 80 μl of 10% APS, 4.8 μl of TEMED, and 3.68 ml of ddH₂O were mixed well. The separating gel was carefully injected into a gel mold with about 2 cm space being left for pouring a spacer gel, and the top layer of the separating gel was covered with absolute ethanol to protect from air and remove bubbles. The gel was allowed to stand at room temperature for about 0.5 h until the separating gel was completely solidified.

5% Spacer Gel (5 ml):

500 μl of the mixed solution of 30% acrylamide, 380 μl of 0.5M tris (pH 6.8), 30 μl of 10% SDS, 30 μl of 10% APS, 3 μl of TEMED, and 2.1 ml of ddH₂O. The 5% spacer gel was prepared immediately before use. The aforementioned materials were mixed evenly, and then injected it into the upper layer of the separating gel without generating bubbles, and a comb was gently inserted into the upper layer.

After the protein was loaded, the spacer gel was kept at a constant pressure of 80 V, after the sample front began to enter the separating gel, the electrophoresis was continued at a constant pressure of 120 V until the color-pre-stained protein molecular weight standard was completely separated.

The separated protein was transferred from the gel onto a PVDF membrane, and subjected to a constant current of 300 A for 2 h.

After membrane transfer, it was soaked in a 10% skimmed milk-TBST blocking buffer, and shaken on a shaker for 2 h to block non-specific binding sites.

The corresponding primary antibodies were diluted with 5% skim milk: HIF-1α was diluted at 1:1000, HIF-2α was diluted at 1:1000, DMT1 was diluted at 1:800, FPN1 was diluted at 1:800, β-actin was diluted at 1:10000, and incubated in the shaker at 4° C. overnight. It was rinsed with the 1×TBST buffer for 3 times, each time for 10 min.

It was added with a secondary antibody (goat anti-rabbit IgG, 1:10000), shaken at room temperature for 1 h, and rinsed with the 1×TBST buffer for 3 times, each time for 10 min.

Color development: the solution A and solution B of the ECL chemiluminescence reagent were mixed in equal volume, added onto the protein surface of the PVDF membrane in an amount of 0.125 mL/cm², and incubated at room temperature for 1 min, and then the mixed solution of A and B.

It was observed with the UVP gel imaging system and photographs were taken.

The intensity of bands was subjected to density analysis with the Vision Works LS image acquisition and analysis software. The experimental results showed the expression level of the target protein as expressed by the ratio of average OD value of the band of the target protein to the band of the internal reference protein β-actin.

### Example 4 Detection by Calcein Loading of Iron Transport-in and Transport-Out Functions in Primary-Cultured Astrocytes and VM Neurons

4.1 Experimental Reagents

Calcein-AM: a product available from Molecular Probes;

6-hydroxydopamine (6-OHDA), ascorbic acid and deferoxamine (DFO): products available from Sigma;

HIF-2α translation inhibitor: a product available from Millipore

4.2 Experimental Instruments

Laser confocal scanning microscope: available from Olympus (IX-81), Japan.

4.3 Liquid Formulation

Ascorbic Acid Solution:

ascorbic acid was dissolved in 0.9% normal saline to obtain a stock solution with a concentration of 200 μg/ml.

6-OHDA:

0.2056 mg of 6-OHDA was taken and dissolved in a ascorbic acid solution to obtain a 1 mM stock solution, and the stock solution was diluted with a serum-free medium to the concentration of a working solution when in use.

HIF-2α Translation Inhibitor:

0.5746 mg of the inhibitor was taken and dissolved in 2 ml of DMSO to obtain a 1 mM stock solution, and the stock solution was diluted with a serum-free medium to the working concentration of 10 μM immediately before use.

HEPES balanced salt solution (HBS): 20 mM HEPES dissolved in 150 mM NaCl, pH=6.8.

Calcein-AM:

1 mg Calcein-AM (MW: 994.87) was dissolved in 1 ml of DMSO to obtain a stock solution with a concentration of 1 mM.

The stock solution was sub-packaged and stored at −20° C., and diluted to a working concentration of 0.5 μM with HBS immediately before use.

Ferrous Sulfate Solution:

2.7802 mg of ferrous sulfate was weighed and dissolved in 10 ml of HBS containing 77.4972 mg of vitamin C (at a molar ratio of 1:44), with the final concentration being 1 mM, and the ferrous sulfate solution was prepared immediately before use.

DFO:

65.68 mg of DFO was weighed and dissolved in 10 ml of HBS, with the final concentration being 1 mM. The DFO solution was prepared immediately before use.

HIF-2α Translation Inhibitor:

immediately before use, it is diluted to a working concentration of 10 μM with a serum-free DMEM/F12 basic medium not containing the double antibodies.

4.4 Inoculation and Drug Treatment of Astrocytes

The primary-cultured astrocytes were plated at a plating density of 5×10⁴/well in a 24-well plate with a polylysine-treated cover slip placed in each well. After the astrocytes were cultured for 48 h, the medium was blotted up, and the cells were rinsed gently with the DMEM/F12 basic medium preheated at 37° C. The cells were then treated in groups with 6 wells in each group, and were continually cultured in a 5% CO₂ incubator at 37° C. for 24 h. The medium was blotted up. The cells were rinsed once with HBS preheated at 37° C., added with calcein (0.5 μM), placed in an incubator at 37° C. for 0.5 h for loading, then rinsed with HBS for three times, and then detected.

4.5 Iron Transport-in and Transport-Out Experiments

Calcein-AM was a membrane-permeable non-fluorescent molecule. It is a membrane-impermeable cytoplasmic dye and a metal-sensitive fluorescent probe after being cleaved into calcein by cytoplasmic esterase in the cells. It was formed by condensation of fluorescein, formaldehyde and iminodiacetic acid, and had strong stability since it was not affected by the pH value of the solution. Calcein had high affinity for all of divalent metal ions, and could be combined with various divalent metal ions in the cells and then converted into non-fluorescent substances, which leads to the quenching of the fluorescence. In this experimental system, other divalent metal ions such as Mg²⁺ and Ca²⁺ were removed, and only a Fe²⁺ solution was used for perfusion to ensure that the fluorescence change of intracellular calcein was caused by its binding with Fe²⁺. The level of cheatable iron in the cells was estimated by determining the decrease degree of the fluorescence intensity. The fluorescence intensity of calcein was inversely proportional to the content of free iron in the cells. When the level of iron in the cells was increased, the free iron combined with calcein, the fluorescence was quenched, and thus the fluorescence intensity was decreased, otherwise it was increased. Therefore, calcein combined with a laser confocal technology could dynamically monitor the level of free iron in the cells.

The cover slip inoculated with cells in the 24-well plate in the above experiment was placed into a tailor-made perfusion tank. Under the action of a peristaltic pump, the perfused liquid can enter the perfusion tank at a constant speed and temperature without affecting the cells on the cover slip. The perfusion tank was placed under a model OlympusIX81 laser confocal microscope, and scanned in a X-Y-Z mode under a 20× objective lens. The wavelength of the excitation light was selected as 488 nm, and the wavelength of the emission light was selected as 525 nm. Firstly, perfusion was carried out with the 1 mM ferrous sulfate solution, and the time-series scanning was set at intervals of 3 min, with a scanning time of 3.5 sec each time, and a total of 10 scans. Then, the perfusion fluid was replaced by HBS preheated at 37° C., which was used for rinsing for 2 min, and finally the HBS was replaced by 1 mM DFO. The scanning settings were the same as those before. The experimental results were analyzed with a software of Fluoview5.0. 4 non-overlapped areas were selected in each visual field, and a total of 25-30 individual cells were selected for fluorescence intensity analysis. Six separate experiments were conducted for each treatment group.

### Example 5 Statistical Treatment

The experimental results were expressed by mean±standard (X±S.E.M), and a SPSS17.0 statistical software was applied. A Student's T test was used for comparison between two groups. Comparison of the data of more than two groups were conducted by One-way ANOVA, followed by comparison of pairwise means by Tukey. P<0.05 showed that the results were statistically significant.

Illustration of Results

1. 6-OHDA Treatment Activates HIF-1α and HIF-2α in Primary Cultured Astrocytes, but not in VM Neurons

To investigate if HIFs are specifically activated in astrocytes treated with 6-OHDA, we used Western blots to quantify HIF-1α and HIF-2α expression in primary cultured astrocytes and VM neurons treated with 6-OHDA. Increased expressions of HIF-1α and HIF-2α was observed in primary cultured astrocytes treated with 10 μM 6-OHDA for 24 h compared to control (FIG. 1a, P<0.01; FIG. 1b, P<0.05). However, protein levels of HIF-1α and HIF-2a remained unchanged in VM neurons after application of 6-OHDA compared to control (FIG. 1c and FIG. 1d; P>0.05). These results indicate that 6-OHDA treatment activates HIF-1α and HIF-2α in primary cultured astrocytes, but not in VM neurons.

2. 6-OHDA-Induced Overexpressions of DMT1 and FPN1 in Cultured Astrocytes do not Require HIF-1α Upregulation

We next tested the effects of BAY 87-2243, a potent and selective HIF-1α inhibitor, on primary cultured astrocytes. Cells were pretreated with 10 μM BAY 87-2243 for 48 h, and then co-treated with 10 μM BAY 87-2243 and 10 μM 6-OHDA for 24 h. Expression of HIF-1α increased in primary cultured astrocytes treated with 10 μM 6-OHDA for 24 h, compared with the control group (FIG. 2a; P<0.05). In contrast, the expression of HIF-1α decreased in primary cultured astrocytes treated with 10 μM BAY 87-2243, compared with the control group (FIG. 2a; P<0.05). Furthermore, BAY 87-2243 pretreatment decreased the expression of HIF-1α in 6-OHDA-treated cells, compared with the 6-OHDA group (FIG. 2a; P<0.05). To further investigate whether HIF-1α modulates the expressions of DMT1 and FPN1 induced by 6-OHDA in primary cultured astrocytes, measured the expressions of DMT1 and FPN1 after inhibiting HIF-1α with BAY 87-2243 (FIG. 2b and FIG. 2c). We observed that DMT1 and FPN1 expression increased in primary cultured astrocytes treated with 10 μM 6-OHDA for 24 h, compared with the control group (FIG. 2b, P<0.01; FIG. 2c, P<0.05). However, the expressions of DMT1 and FPN1 remained unchanged in astrocytes treated with 10 μM BAY 87-2243 alone, compared with the control group (FIG. 2b; FIG. 2c; P>0.05). Expressions of DMT1 and FPN1 did not change in the BAY 87-2243 pretreatment group, compared to the 6-OHDA group (FIG. 2B; FIG. 2C; P>0.05). Overall, these results demonstrate that BAY 87-2243 suppressed expression of HIF-1α and inhibited the overexpression of HIF-1α in primary cultured astrocytes treated with 6-OHDA. However, BAY 87-2243 did not inhibit the up-regulation of DMT1 and FPN1 by 6-OHDA, suggesting that HIF-1α does not contribute to these processes.

3. HIF-2α Upregulation is Required for 6-OHDA Induced Overexpressions of DMT1 and FPN1

We next investigated the effects of a potent and selective inhibitor of HIF-2a, which acts specifically on the subunits of HIF-2a. Primary cultured astrocytes were pretreated with 10 μM HIF-2α inhibitor for 24 h, and then co-treated with 10 μM HIF-2α inhibitor and 10 μM 6-OHDA for 24 h. Expression of HIF-2α increased in primary cultured astrocytes treated with 10 6-OHDA for 24 h, compared with the control group (FIG. 3a; P<0.05). In contrast, treatment with 10 μM HIF-2α inhibitor decreased the expression of HIF-2α in primary cultured astrocytes, compared with the control group (FIG. 3a; P<0.05). Furthermore, HIF-2α inhibitor pretreatment decreased the expression of HIF-2α in 6-OHDA-treated cells, compared with the 6-OHDA group (FIG. 3a; P<0.01). To obtain direct evidence about whether HIF-2α contributes to modulating the expression of DMT1 and FPN1 in activated primary cultured astrocytes, we measured expression levels of DMT1 and FPN1 in primary cultured astrocytes treated with HIF-2α inhibitor. Expressions of DMT1 and FPN1 increased in primary cultured astrocytes treated with 10 μM 6-OHDA for 24 h, compared with the control group (FIG. 3b, P<0.05; FIG. 3c, P<0.01). However, DMT1 and FPN1 expression decreased in primary cultured astrocytes treated with 10 μM HIF-2α inhibitor, compared to the control group (FIG. 3b, P<0.01; FIG. 3c, P<0.05). Similarly, HIF-2α inhibitor pretreatment decreased the expressions of DMT1 and FPN1 in 6-OHDA-treated cells, compared with the 6-OHDA group (FIG. 3b, P<0.05; FIG. 3c, P<0.01). In summary, the HIF-2α inhibitor suppressed expression of HIF-2a, as expected, and prevented the overexpression of HIF-2α in primary cultured astrocytes by 6-OHDA. These results suggest that HIF-2α modulates the influence of 6-OHDA on DMT1 and FPN1 expression.

4. HIF-2α was Involved in the Increased Ferrous Iron Influx and Efflux in 6-OHDA-Treated Primary Cultured Astrocytes

The fluorescent dye calcein was used to monitor ferrous iron influx into primary astrocytes during a 1 mM ferrous iron perfusion. The fluorescence intensity declined gradually inside cells, indicating a transmembrane ferrous iron influx (FIG. 4). We investigated the roles of astrocytes in regulating iron homeostasis under oxidative conditions. In agreement with our previous data for primary astrocytes(H. Y. Zhang et al., 2013), cells treated with 10 μM 6-OHDA showed a more rapid fluorescence quenching and a decrease in fluorescence intensity compared with controls. However, pretreatment with 10 μM HIF-2α inhibitor for 24 h fully blocked this process and produced a fluorescence intensity similar to control, indicating that the HIF-2α inhibitor suppresses the increased ferrous iron influx caused by 6-OHDA.

For the iron efflux assay, we used DFO, a membrane-impermeable, potent, specific iron chelator. When cells were perfused with 1 mM DFO, a marked increase in fluorescence intensity was observed in the 10 μM 6-OHDA-treated group. These results indicate that 6-OHDA enhances iron transportation rate in primary cultured astrocytes. However, pretreatment with 10 μM HIF-2α inhibitor for 24 h fully blocked this process and yielded a fluorescence intensity similar to control levels, indicating that the HIF-2α inhibitor suppresses the ferrous iron efflux caused by 6-OHDA.

1. Hypoxia-inducible factor-2a as an action target in prevention/treatment of the PD.

2. The use according to claim 1, comprising the following steps:
respectively treating primary-cultured mesencephalon astrocytes and primary-cultured mesencephalon ventral neurons with 6-OHDA, respectively detecting the protein expressions of HIF-1α and HIF-2α in the extracted total proteins, and respectively comparing with the protein expressions of HIF-1α and HIF-2α in a group not treated with 6-OHDA, wherein HIF-1α and HIF-2α are detected in the astrocytes activated with 6-OHDA, and are not detected in the non-activated mesencephalon ventral neurons, so that it is confirmed that in a PD cell model prepared with 6-OHDA, 6-OHDA activates HIF-1α and HIF-2α in the primary-cultured mesencephalon astrocytes;
using Bay87-2243 as HIF-1α specific inhibitor to act on the primary-cultured mesencephalon astrocytes, then using 6-OHDA for co-action, and observing the change in the protein expression of HIF-1α in the astrocytes and the change in the protein expressions of DMT1 and FPN1 in the astrocytes after HIF-1α activation is inhibited;
using HIF-2α translation inhibitor as HIF-2α specific inhibitor to act on the primary-cultured mesencephalon astrocytes, then using 6-OHDA for co-action, and observing the change in the protein expression of HIF-2α in the astrocytes and the change in the protein expressions of DMT1 and FPN1 in the astrocytes after HIF-2α activation is inhibited; and
screening based on the aforementioned changes in the protein expression in the primary-cultured mesencephalon astrocytes and primary-cultured mesencephalon ventral neurons, to obtain HIF-2α as an action target for prevention/treatment of PD.

3. The use according to claim 2, wherein the activation of HIF-2α in the primary-cultured astrocytes can cause up-regulation of the protein expressions of DMT1 and FPN1, and the inhibition of HIF-2α in the astrocytes can also inhibit the up-regulation of the protein expressions of DMT1 and FPN1.

4. The use according to claim 2, wherein the HIF-1α inhibitor inhibits the protein expression of HIF-1α in the primary-cultured astrocytes, but does not inhibit the up-regulation of the protein expressions of DMT1 and FPN1 in the primary-cultured astrocytes as induced by 6-OHDA.

5. The use according to claim 2, wherein the primary-cultured astrocytes and the primary-cultured mesencephalon ventral neurons are respectively treated with 10 μM of 6-OHDA and then inoculated in a 6-well plate pre-coated with polylysine at a density of 1.5×10⁵/ml, and then 24 h later the total protein is extracted respectively; and
pretreatment is conducted for 48 h or 24 h with 10 μM of the HIF-1α specific inhibitor Bay87-2243 or the HIF-2α translation inhibitor as the HIF-2α specific inhibitor in connection with instructions of the inhibitors and a screen-time experiment, then added with 10 μM of 6-OHDA for co-action for 24 h, and then the total protein is extracted.

6. The use according to claim 5, wherein the method for extracting and quantifying the total protein comprises the following steps:
treating cells with 6-OHDA or the HIF-1α or HIF-2α specific inhibitor, blotting up the culture medium, washing with pre-chilled PBS, adding 100 μl of RIPA cell lysis buffer into each well, and lysing on ice for 0.5 h to obtain a lysate; and
collecting the lysate, then centrifuging at 4° C. and 12000 rpm for 20 min, and transferring the supernatant into a new EP tube; detecting the concentration of the extracted protein with a BCA protein quantitative kit, sub-packaging the protein according to 30 μg per serving, heating in a water bath at 95° C. for 5 min to denature the protein, and cryopreserving in a refrigerator at −80° C.

7. The use according to claim 2, wherein the culture approach of the astrocytes comprises: extracting and separating the primary mesencephalon astrocytes from a test animal, resuspending the precipitate, screening, then digesting with pancreatin, and terminating the digestion with a astrocyte complete medium, then adding the astrocyte complete medium for differential adhesion treatment, adjusting the cell concentration, then inoculating into a 6-well plate pre-treated with polylysine, and stabilizing for 24-48 h for experiments;
the culture approach of the mesencephalon ventral neurons comprises: extracting and separating the primary mesencephalon ventral neurons from a test animal, inoculating into a six-well plate pre-treated with polylysine, placing in a 5% CO₂ incubator at 37° C. for 5 days, and then using for experiments.

8. The use according to claim 7, wherein the astrocyte complete medium consists of 200 ml of a DMEM/F-12(1:1) basic medium, 10% fetal bovine serum, 100 U/ml of penicillin and 0.1 mg/ml of streptomycin.

9. The use according to claim 7, wherein a mesencephalon ventral inoculation medium consists of 200 ml of the DMEM/F-12(1:1) basic medium, 10% fetal bovine serum, 100 U/ml of penicillin and 0.1 mg/ml of streptomycin; and a mesencephalon ventral neuron complete medium consists of 200 ml of the DMEM/F-12(1:1) basic medium, 2% B27, 100 U/ml of penicillin and 0.1 mg/ml of streptomycin.

10. The use according to claim 1, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

11. The use according to claim 2, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

12. The use according to claim 3, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

13. The use according to claim 4, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

14. The use according to claim 5, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

15. The use according to claim 6, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

16. The use according to claim 7, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

17. The use according to claim 8, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.

18. The use according to claim 9, wherein the pathological feature of the PD is the loss of dopaminergic neurons in SNpc, iron deposition of the PD is mainly concentrated in the DA neurons, and the iron deposition in the DA neurons leads to degeneration and death of the DA neurons, leading to the occurrence of the PD.
